(19) United States
(12) Patent Application Publication
(10) Pub. No.: US 2005/0010252 A1
(43) Pub. Date: Jan. 13, 2005

(54) Methods, systems and computer program products for defibrillation by applying stimulus to a fastest activating region of a heart

(76) Inventor: Raymond E. Ideker, Birmingham, AL (US)

Correspondence Address: MYERS BIGEL SIBLEY & SAJOVEC, PO BOX 37428, RALEIGH, NC, 27627, US

(21) Appl. No.: 10/615,528

(22) Filed: Jul. 8, 2003

(51) Int. Cl. A61N 1/39

(52) U.S. Cl. 607/5; 607/4

(57) ABSTRACT

Methods for defibrillating a heart in fibrillation can include detecting fibrillation of the heart and applying a defibrillation stimulus to a fastest activating region of the fibrillating heart. Applying electrical stimuli to the region of the heart containing the fastest activating region may cause the “mother rotor” to halt, thereby interrupting fibrillation of the heart. Alternatively, applying the electrical stimuli to a region of a heart not in fibrillation but is likely to include the mother rotor during fibrillation may reduce the likelihood that fibrillation may occur in the heart.

### STATEMENT OF GOVERNMENT SUPPORT

This invention was made with government support under National Institutes of Health grant number HL-66256. The government has certain rights to this invention.

### FIELD OF THE INVENTION

The present invention relates generally to the field of treatments for cardiac airhythmia and, more particularly, to defibrillation of hearts.

### BACKGROUND

Rotating electrical waves, commonly referred to as rotors, are considered possible “organizing centers” for fibrillation of the heart. In contrast, a refractory period associated with tissue in the heart is considered a secondary factor which may modulate the location of these rotors, but may not be considered to be the primary force which sustains the fibrillation.

Electrical and optical mapping have demonstrated some organization during fibrillation (i.e. ventricular fibrillation (VF) or atrial fibrillation (AF)). In particular, activation fronts have been shown to be large during fibrillation and follow pathways that are centimeters in length. In addition, many of these activation fronts may follow similar pathways. However, after the first few seconds of VF, rotors may dissipate and be difficult to observe. In some mapping studies, 2% to 8% of activation fronts were identified as parts of reentrant circuits. In addition, most of the reentrant circuits were observed to be short-lived, lasting typically slightly more than one cycle.

One proposed mechanism for fibrillation has focused on rotors as transient, unstable objects, and VF is sometimes explained in terms of how rotors can break up to form the turbulent state seen in epicardial maps. In another proposed mechanism, a “mother rotor” may break up into “daughter rotors,” which in turn may break up in a continual chain of succession. Individual daughter rotors may or may not complete a full cycle of reentry. One proposed breakup mechanism is sometimes called the restitution hypothesis which states that when the slope of the Action Potential Duration (APD) restitution curve is greater than 1, then an APD at a particular site will undergo beat-to-beat oscillations, culminating in localized block and possibly create a reentrant circuit.

In Zaitsev A V, et al. Distribution of excitation frequencies on the epicardial and endocardial surfaces of fibrillating ventricular wall of the sheep heart. Circ. Res. 2000;86;408-417, sustained reentry was uncommonly observed, even in the fastest-activating domain. Instead, activation fronts were observed to break through to the epicardial and endocardial surfaces, as has been observed by others. Zaitsev, however, proposes that the mother rotor in the fastest activating region is intramural. Mother rotors in the relation to fibrillation are also discussed in Mechanisms of Atrial Fibrillation: Mother Rotors or Multiple Daughter Wavelets, or Both? by Jose Jalife, M. D. et al., J Cardiovascular Electrophysiology. 1998;9(suppl):S2-S12.

### SUMMARY

Embodiments according to the present invention can provide methods, systems, and computer program products for interrupting or reducing the likelihood of fibrillation of the heart. Pursuant to these embodiments, methods for defibrillating a heart in fibrillation can include detecting fibrillation of the heart and applying a defibrillation stimulus to a fastest activating region of the fibrillating heart.

Applying electrical stimuli to the region of the heart containing the fastest activating region may cause the “mother rotor” to halt, thereby interrupting fibrillation of the heart. Alternatively, in some embodiments according to the present invention, applying the electrical stimuli to a fastest activating region can reduce the likelihood of fibrillation occurring or halt fibrillation which is already occurring.

In some embodiments according to the present invention, the fastest activating region can be a reentrant region having a refractory period that is less than areas adjacent to the reentrant region. In some embodiments according to the present invention, a first wavefront propagates along a closed pathway on the fibrillating heart, wherein the first wavefront generates at least a second wavefront that propagates on the fibrillating heart outside the fastest activating region. In some embodiments according to the present invention, the wavefront propagates along the closed pathway from a starting point on the closed pathway to an ending point on the closed pathway.

### BRIEF DESCRIPTION OF THE DRAWINGS

FIG. 1 is a schematic diagram of a heart showing a mother rotor included in a fastest activating region of the heart.

FIG. 2 is a schematic illustration of a heart including a mother rotor in a fastest activating region of the heart coupled to a defibrillator according to embodiments of the present invention.

FIG. 3 is a flowchart illustrating method embodiments according to the present invention.

FIG. 4 is a schematic illustration of a heart including a mother rotor in a fastest activating region of the heart coupled to a defibrillator circuit and a pacing circuit according to embodiments of the present invention.

FIG. 5 is a schematic illustration of a heart coupled to a processor circuit that can be used to determine a fastest activating region of a heart according to embodiments of the present invention.

### DESCRIPTION OF EMBODIMENTS ACCORDING TO THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, components or features may be exaggerated for clarity. It will be understood that the term “a,” when referring to an element, includes a single element and at least one element.

As will be appreciated by one of skill in the art, the present invention may be embodied as a method, data processing system, or computer program product. Accordingly, the present invention may take the form of an entirely hardware embodiment, an entirely software embodiment or an embodiment combining software and hardware aspects, all generally referred to herein as a “circuit.” Furthermore, the present invention may take the form of a computer program product on a computer-usable storage medium having computer-usable program code means embodied in the medium. Any suitable computer readable medium may be utilized including, a memory device, hard disks, CD-ROMs, optical storage devices, a transmission media, such as a wireless transmission media and/or those supporting the Internet or an intranet, or magnetic storage devices.

The present invention is described herein with reference to a flowchart and block diagrams of methods, apparatus (systems) and computer program products according to embodiments of the invention. It will be understood that each block, and combinations of blocks, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the block or blocks.

While embodiments of the present invention are described with reference to a particular architecture and/or division of functions, the present invention should not be construed as limited to such architecture and/or division. Thus, other architectures and/or division of functions capable of carrying out the operations described herein may be utilized while still falling within the teachings of the present invention. Furthermore, while embodiments of the present invention are described with reference to particular circuits, such circuits may include discrete components, processors, such as a microprocessor and/or signal processor, analog circuits, digital circuits and/or combinations thereof.

With regard to the operations illustrated in the flowchart, as will be appreciated by those of skill in the art in light of the present disclosure, embodiments of the present invention are not limited to the specific sequence or sequences of operations described therein. Thus, for example, operations in the flowchart may be provided out of sequence or concurrently. Similarly, other sequences of operations may be utilized while still providing the feedback adjustment according to embodiments of the present invention. Accordingly, the present invention should not be construed as limited to the particular operations or sequence of operations illustrated in the flowchart.

Embodiments according to the present invention may be used to interrupt, or reduce the likelihood of, fibrillation in hearts by, for example, applying electrical stimuli to a region of the heart that contains what is sometimes referred to as the “mother rotor.” Applying electrical stimuli to the region of the heart containing the fastest activating region may cause the “mother rotor” to halt, thereby interrupting fibrillation of the heart. Alternatively, in some embodiments according to the present invention, applying the electrical stimuli to a region that is likely to include the mother rotor during fibrillation may reduce the likelihood that fibrillation may occur in the heart. As used herein, the term fibrillation can include both atrial fibrillation as well as ventricular fibrillation.

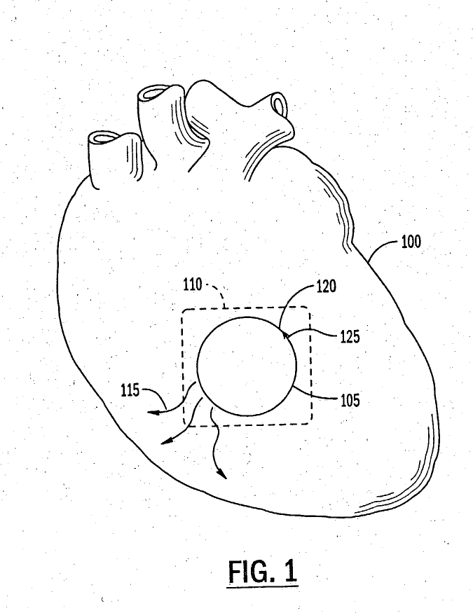

FIG. 1 is a schematic illustration of a heart 100 including a mother rotor 105 occurring in a fastest activating region 110 of the heart 100. The fastest activating region 110 can be a region of the heart 100 which activates faster than adjacent regions of the heart 100 when the heart 100 is in fibrillation. In other words, the fastest activating region 110 can be a region of the heart 100 which has a refractory period which is shorter than adjacent regions of the heart 100. As will be understood by those having skill in the art, the refractory period of tissue is sometimes used to describe the minimum time after which the tissue is excited that the same tissue can be subsequently excited. For example, if the refractory period of a tissue is tᵣ, after causing the tissue to contract in response to a first stimulus, a subsequent stimulus will only cause the tissue to activate a second time if the second stimulus is applied at least tᵣ after the first stimulus. If the second stimulus is applied prior to tᵣ, the tissue may not activate in response to the second stimulus. For example tᵣ can be in a range of about 0.5 seconds to about 0.05 seconds. Therefore, the fastest activating region 110 can be a region of the heart 100 which has the shortest refractory period needed to maintain fibrillation of the heart 100. Although a single mother rotor 105 is shown in FIG. 1, it will be understood that multiple mother rotors 105 may exist within the heart 100.

According to FIG. 1, the mother rotor 105 can be a wavefront which propagates through tissue in the fastest activating region 110 over a pathway that originates at a start point 120 and ends at an end point 125 that is immediately adjacent to the start point 120. Although the pathway is shown as having a circular shape, it will be understood by those having skill in the art that the pathway followed by the mother rotor 105 can have any other shape, such as shapes that are defined by an irregular pathway through the tissue. It will be further understood that the wavefront that defines the mother rotor 105 propagates from the start point 120 to the end point 125 over the pathway in a time which is greater than or equal to the refractory period of the tissue included along the pathway that defines the mother rotor 105 as discussed herein. For example, if the refractory period is tᵣ the wavefront that defines the mother rotor 105 propagates from the start point 120 to the end point 125 in a time interval which is greater than or equal to tᵣ.

It will also be understood that mother rotor 105 can be an idealized version of a wave front that propagates through the fastest activating region 110. In particular, a wave front that propagates through the fastest activating region can vary in time and space as compared to the idealized version depicted by the mother rotor 105. For example, a wave front propagating in the fastest activating region may travel along different paths during different cycles of the propagation. Furthermore, the period required for a wave front to complete one cycle may vary over time. A wave front that travels through the fastest activating region can vary from the idealized version of the idealized version of the mother rotor in other ways as well. It will be further understood that more than one wavefront may be propagating in the fastest activating region wherein one of the wavefronts is a mother rotor.

Because the wavefront which defines the mother rotor 105 travels from the start point 120 to the end point 125 in a time that is greater than or equal to the shortest refractory period, the mother rotor 105 can be self sustaining. In particular, if the wavefront arrives at the end point 125 at least tᵣ after the wavefront began to propagate from the start point 120, the tissue at the start point 120 can be excited again in response to the wavefront. Therefore, the wavefront that defines the mother rotor 105 can reenter the start point 120 from the end point 125 so that the mother rotor 105 propagates over substantially the same pathway through the tissue multiple times. It will be understood to those skilled in the art that the propagation of the wavefront described herein can result in the region 110 being described as being “re-entrant” in nature. The mother rotor 105 can cause daughter waves 115 that emanate from the mother rotor 105 which can create/maintain the occurrence of fibrillation in the heart 100. According to embodiments of the present invention, applying a defibrillation stimulus to the fastest activating region (i.e., the region containing the mother rotor 105) can cause the mother rotor 105 to be interrupted thereby interrupting the daughter waves 115 which may halt the fibrillation of the heart 100.

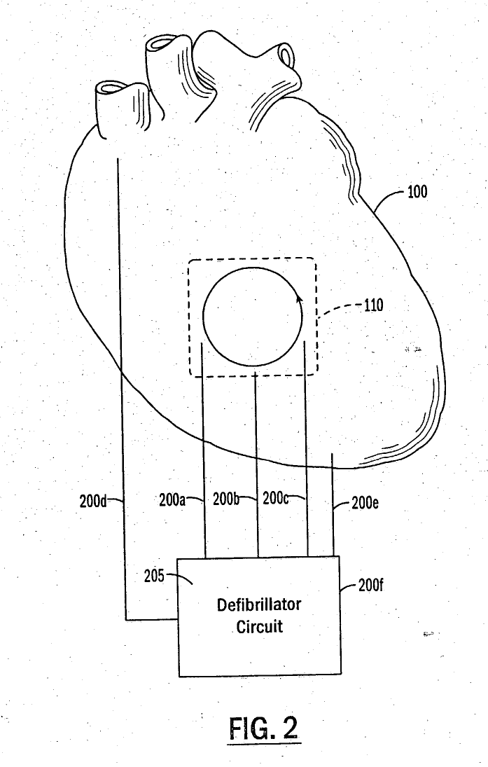

FIG. 2 is a schematic view of a heart that illustrates a defibrillator circuit 205 that is electrically coupled to the fastest activating region 110 of a fibrillating heart 100 in fibrillation via electrodes 200a-c. According to FIG. 2, the defibrillator circuit 205 is configured to apply the defibrillation stimulus to the fastest activating region 110 of the fibrillating heart 100 via the electrodes 200a-c. In some embodiments according to the present invention, the fastest activating region 110 is located at a base of the left ventricle of the fibrillating heart 100. In other embodiments according to the present invention, the fastest activating region 110 is located in the septum of the fibrillating heart 100 when the heart is in ventricular fibrillation. In some embodiments according to the present invention, such as when the heart 100 is in atrial fibrillation, the fastest activating region 110 can be located adjacent to the pulmonary veins or between a pulmonary vein and the left atrial appendage of the fibrillating heart 100. In some embodiments according to the present invention, an electrode can be inserted through the great cardiac vein and the coronary sinus (at the base of the left ventricle). In other embodiments according to the present invention, an electrode can be inserted in the right ventricle of the septum up against the right side. In each of these embodiments according to the present invention, the electrodes 200a-c are electrically coupled to the respective region of the heart 100 that includes the fastest activating region 110.

Although the electrodes are described as being electrically coupled to respective regions of the heart (such as the fastest activating region 110), it will be understood that the electrodes may be coupled to regions which are proximate to the fastest activating region so that the stimuli provided has the desired effect. For example, in some embodiments according to the present invention, the electrode can be applied to the epicardial or pericardial surfaces of the heart when the fastest activating region may be located intramurally. Therefore, when an electrical stimuli is described as being applied to a particular region, it will be understood that the electrode through which the electrical stimulus is applied can be spaced apart from the region to which the stimulus is applied yet proximate enough to deliver the stimulus to the intended region to have the desired effect.

In some embodiments according to the present invention, the defibrillator circuit 205 can apply a single defibrillation stimulus using multiple electrodes shown in FIG. 2. In some embodiments according to the present invention, both electrodes used to provide the single defibrillation stimulus are located inside the fastest activating region. In other embodiments according to the present invention, one of the electrodes is located inside the fastest activating region and the other electrode is located outside the fastest activating region. For example, a single defibrillation stimulus can be applied using a first electrode 200a located inside the fastest activating region 110 and a second electrode located outside the fastest activating region, such as electrodes 200d, 200e, and 200f. It will be understood by those skilled in the art, that electrode 200f can represent the exterior housing of the defibrillator circuit 205 (i.e. a hot CAN). It will be further understood that multiple electrodes can be used in conjunction with one another to function as a single electrode. For example, the two electrodes 200d and 200e can serve as either the cathode electrode or as the anode electrode in providing a defibrillation stimulus. It will be further understood that any combination of electrodes can be used in conjunction with one another to provide a single one of the electrodes used to provide the defibrillation stimulus.

In some embodiments according to the present invention, the defibrillator circuit 205 can apply a series of defibrillation stimuli. For example, in some embodiments according to the present invention, the defibrillator circuit 205 can apply a first defibrillation stimulus and a second subsequent defibrillation stimulus. In particular, the first defibrillation stimulus can precede or follow the second defibrillation stimulus which can have a greater magnitude than the first defibrillation stimulus. In still other embodiments according to the present invention, the first defibrillation stimulus can also be applied simultaneously with the second defibrillation stimulus.

In some embodiments according to the present invention, the first and second defibrillation stimuli can be applied using different electrodes as discussed above in reference to the use of a single defibrillation stimulus. For example, in some embodiments according to the present invention, the first defibrillation stimulus can be applied via a first set of electrodes, such as 200a-b, whereas the second defibrillation stimulus can be applied by a second set of electrodes, such as 200b-c. It will be understood that the stimuli can also be applied using other combinations of the electrodes 200a-c.

In some embodiments according to the present invention, some of the electrodes that are outside the fastest activating region can also be used to provide the defibrillation stimuli. For example, in some embodiments according to the present invention, the electrodes 220d, 200e, and 200f that are outside the fastest activating region 110, operating in conjunction with the electrodes 220a, 200b and 200c that are located inside the fastest activating region, can be used to provide the stimuli. As discussed above, it will also be understood that some of the electrodes can function as a single electrode. For example, electrodes 200d, 200e, and 200f can operate as a single electrode in conjunction with one (or a combination of) the electrodes 200a, 220b and 200c that are located inside the fastest activating region 110. It will be further understood, that in some embodiments according to the present invention, the defibrillation stimuli can be applied as discussed above in reference to the application of a single defibrillation stimulus.

It will further be understood that although FIG. 2 shows three electrodes electrically coupled to the fastest activating region 110, additional or fewer electrodes can be used. In some embodiments according to the present invention, a single electrode such as electrode 200c, can be electrically coupled to the fastest activating region while one or more other electrodes can be electrically coupled to regions of the heart that are outside the fastest activating region using, for example, electrodes 200d and 200e to apply the defibrillation stimulus. It will further be understood that the defibrillator circuit 205 can be an implantable defibrillator such as an implantable ventricular defibrillator, an implantable atrial defibrillator, or an implantable atrial/ventricular defibrillator.

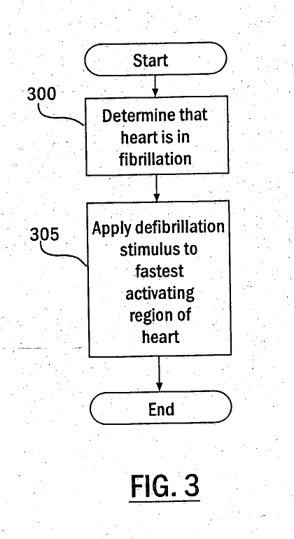

FIG. 3 is a flowchart that illustrates method embodiments according to the present invention. In particular, in embodiments according to the present invention, where for example fibrillation of the heart is to be halted, a determination is made that the heart is currently in fibrillation (block 300). It will be understood by those skilled in the art that the determination of whether the heart is in fibrillation can be done using conventional techniques. After the heart is determined to be in fibrillation, the defibrillation stimulus (or stimuli) is applied to the fastest activating region of the heart (block 305) as discussed above in reference to FIG. 2. Furthermore, as discussed above, the stimulus may be applied at different times and/or via different electrodes coupled to the heart 100.

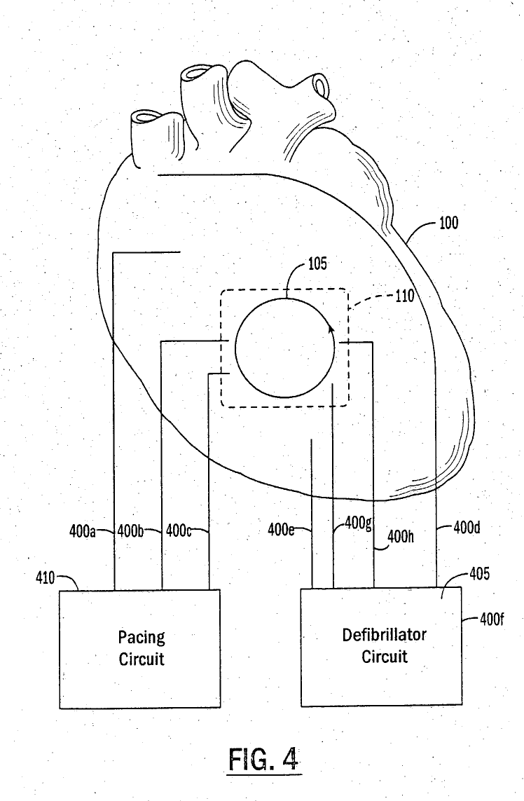

FIG. 4 is a schematic view of the heart 100 and a pacing circuit 410 electrically coupled to the heart 100 by electrodes 400a-c and a defibrillator circuit 405 electrically coupled to the heart 100 by electrodes 400d-e according to embodiments of the present invention. In some embodiments according to the present invention, the pacing circuit 410 can be used to apply at least one pacing stimulus to the fibrillating heart 100 in conjunction with the defibrillation stimulus applied by defibrillator circuit 405. For example, as discussed above in reference to FIGS. 2 and 3, defibrillator circuit 405 can apply defibrillation stimuli to the fastest activating region 110 via electrodes 400g-h. The pacing circuit 410 can be configured to apply at least one pacing stimulus to the fastest activating region 110 of the fibrillating heart 100, for example, via electrodes 400b-c. In some embodiments according to the present invention, the pacing circuit 410 can be configured to apply the pacing stimulus to a region of the fibrillating heart 100 which is spaced apart from the fastest activating region 110. In some embodiments according to the present invention, the pacing stimulus can be applied to the fastest activating region 110 (via electrode 400b or c), and the defibrillation stimulus can be applied outside the fasting activating region 110, such as in the superior vena cava or in the right ventricle via electrodes 400e and 400d, or in other regions outside the fasting activating region 110 using, for example, the exterior housing of the defibrillator circuit 405 as an electrode 400f.

In operation, the pacing circuit 410 can be configured to apply pacing stimuli before, during, or after stimuli applied by the defibrillator circuit 405. In some embodiments according to the present invention, pacing stimuli can be used to increase the efficacy of the defibrillation stimuli applied by the defibrillator circuit 405a. For example, in some embodiments according to the present invention, a determination can be made as to the location of the fastest activating region. Pacing stimuli can then be applied to the fastest activating region of the heart. Once the pacing stimuli has been applied to the fastest active region, ceasing the pacing stimuli may be adequate to interrupt fibrillation. In other embodiments according to the present invention, pacing can be used prior to the occurrence of fibrillation by, for example, sensing the occurrence of nonsustained arrhythmia for a number of beats of the heart followed by pacing in the fastest activating region. In some embodiments according to the present invention, the pacing stimuli can be about 5 Volts and the defibrillation stimuli can be about 100 Volts. In some embodiments according to the present invention, the pacing stimuli can be about 20 Volts and the defibrillation stimuli can be about 1000 Volts.

In other embodiments according to the present invention, the electrodes can be those disclosed in U.S. Pat. No. 5,873,896 to Ideker entitled Cardiac device for reducing arrhythmia, the disclosure of which is incorporated herein by reference in its entirety.

In other embodiments according to the present invention, electrical stimuli can be utilized to reduce the likelihood that fibrillation will occur. In some embodiments according to the present invention, a premature contraction of the heart is detected over a plurality of beats, such as occurs in nonsustained tachycardia. Upon such detection, a defibrillation stimulus is applied to the region of the heart at a fastest activating region in which a mother rotor may occur during fibrillation. In some embodiments according to the present invention, the region of the heart that contains the fastest activating region can be assumed. For example, in some embodiments according to the present invention, the fastest activating region can be assumed to arise in regions such as the base of the left ventricle, in the septum, adjacent to the pulmonary veins, or between the pulmonary vein and a left atrial appendage. The fastest activating region may also occur in other regions.

In some embodiments according to the present invention, the fastest activating region of the heart can be determined based on measurement. For example, in some embodiments according to the present invention, techniques known to those having skill in the art can be used to determine (in the absence of fibrillation) which regions are likely to contain the fastest activating region of the heart during fibrillation, such as Monophasic Activation Potential (MAP) electrode readings and activation recovery interval measurements, or premature stimulation to determine refractory periods.

In other embodiments according to the present invention, fibrillation of the heart can be induced to determine where the fastest activating region is likely to be located during subsequent episodes of fibrillation. For example, the fastest activating region can be determined using premature stimulation of the heart after inducing fibrillation.

In some embodiments according to the present invention, the fastest activating region of the heart can be determined based on measurements while the heart is in ventricular fibrillation. For example, in some embodiments according to the present inventions, the number of activations per second over a region of tissue in the heart can be counted to determine which region exhibits the most activations per second (i.e. the fastest activating region.) In other examples according to the present invention, a power spectrum analysis can be performed for regions of the heart wherein the regions having a spectrum with the peak power at the highest frequency can indicate which region is the fastest activating region of the heart.

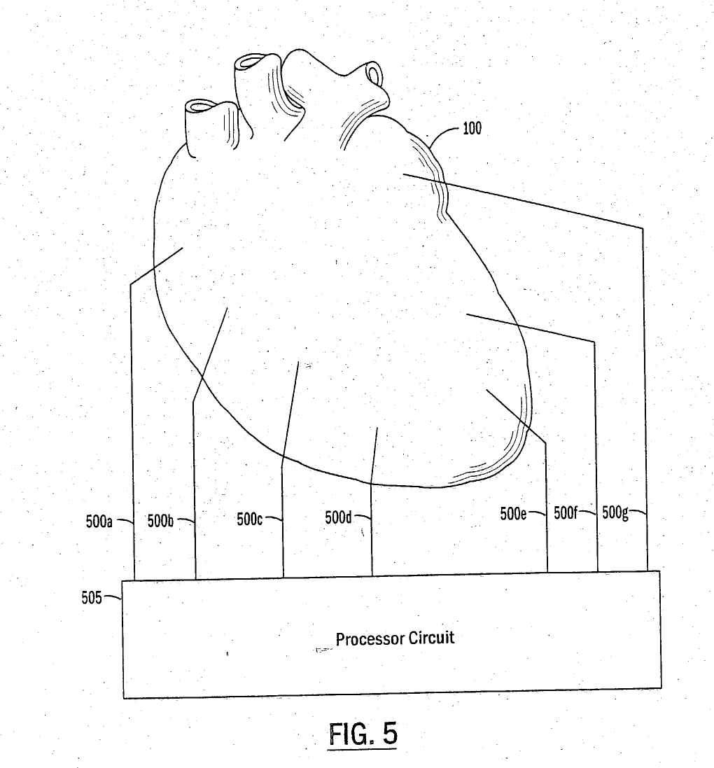

In some embodiments according to the present invention, electrodes can be attached to the heart 100 as shown in FIG. 5. As shown in FIG. 5, electrodes 500a-g can be applied to various regions of the heart 100 which can be used to monitor activity in the heart 100, for example, after fibrillation is induced. In some embodiments according to the present invention, the monitoring of the various regions can be provided by moving one or more of the electrodes to the desired regions using, for example, a catheter. A processor circuit 505 can be used to process electrical signals provided thereto via the electrodes 500a-g to determine which region is the fastest activating region. Subsequently, a defibrillator circuit, such as an implantable defibrillator, can be electrically coupled to the region determined to be the fastest activating region.

Although a few exemplary embodiments of this invention have been described, those skilled in the art, having the benefit of this disclosure, will appreciate that many modifications are possible without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method for defibrillating a heart in fibrillation, comprising:
detecting fibrillation of the heart; and
applying a defibrillation stimulus to a fastest activating region of the fibrillating heart.

2. A method according to claim 1 wherein the fastest activating region comprises a reentrant region having a refractory period that is less than areas adjacent to the reentrant region.

3. A method according to claim 1 wherein a first wavefront propagates along a closed pathway on the fibrillating heart, wherein the first wavefront generates at least a second wavefront that propagates on the fibrillating heart outside the fastest activating region.

4. A method according to claim 1 wherein the fastest region comprises a closed pathway on the fibrillating heart.

5. A method according to claim 4 wherein a wavefront propagates along the closed pathway from a starting point on the closed pathway to an ending point on the closed pathway.

6. A method according to claim 5 wherein the starting point and the ending point are adjacent to one another on the closed pathway.

7. A method according to claim 1:
wherein the fibrillation comprises atrial fibrillation; and
wherein the fastest activating region comprises at least one of adjacent to pulmonary veins of the fibrillating heart and between the pulmonary veins and a left atrial appendage of the fibrillating heart.

8. A method according to claim 1:
wherein the fibrillation comprises ventricular fibrillation; and
wherein the fastest activating region comprises a base of a left ventricle of the fibrillating heart.

9. A method according to claim 1:
wherein the fibrillation comprises ventricular fibrillation; and
wherein the fastest activating region comprises a septum of the fibrillating heart.

10. A method according to claim 1 wherein applying comprises applying a first defibrillation stimulus at least one of before, during, or after a second defibrillation stimulus that is greater than the first defibrillation stimulus.

11. A method according to claim 10 wherein at least one of the first and second defibrillation stimuli is applied using at least a pair of electrodes, wherein the pair of electrodes is located one of:
inside the fastest activating region;
outside the fastest activating region; and
a first electrode of the pair of electrodes is inside the fastest activating region and a second electrode of the pair of electrodes is outside the fastest activating region.

12. A method according to claim 1 wherein the defibrillation stimulus is applied using a pair of electrodes, wherein the pair of electrodes is located one of:
inside the fastest activating region;
outside the fastest activating region; and
a first electrode of the pair of electrodes is inside the fastest activating region and a second electrode of the pair of electrodes is outside the fastest activating region.

13. A method according to claim 1 further comprising:
applying at least one pacing stimulus to the fastest activating region simultaneously with the defibrillation stimulus.

14. A method according to claim 1 further comprising:
applying at least one pacing stimulus to the fastest activating region immediately before or after the defibrillation stimulus.

15. A method according to claim 1 further comprising:
applying at least one first pacing stimulus at the fastest activating region at least one of before, simultaneous with, or after the defibrillation stimulus; and
applying at least one second pacing stimulus to the fibrillating heart at a location spaced-apart from the fastest activating region.

16. A method according to claim 16 wherein the at least one second pacing stimulus is applied using at least one line electrode.

17. A method according to claim 1 wherein the fastest activating region comprises the septum.

18. A method according to claim 1 wherein a location of the fastest activating region is determined by:
determining a monophasic activation potential (MAP) reading associated with the fibrillating heart.

19. A method for reducing an occurrence of fibrillation of a heart, comprising:
detecting a premature contraction of the heart for a plurality of heart beats characterized by nonsustained tachycardia; and
applying an electric stimulus to a region of the heart that is likely to contain a fastest activating region.

20. A method according to claim 19 wherein the electric stimulus comprises one of a defibrillation stimulus and a pacing stimulus.

21. A method according to claim 19 wherein a location of the fastest activating region is determined by:
inducing fibrillation of the heart; and
determining at least one of a monophasic activation potential (MAP) reading associated with the fibrillating heart, a refractory period associated with the heart using premature stimulation, and a power spectrum analysis to provide a spectrum with a peak power at a highest frequency.

22. A method according to claim 19 wherein the fastest activating region comprises a reentrant region having a refractory period that is less than areas adjacent to the reentrant region.

23. A method according to claim 19 wherein a first wavefront propagates along a closed pathway on the fibrillating heart, wherein the first wavefront generates at least a second wavefront that propagates on the fibrillating heart outside the fastest activating region.

24. A method according to claim 19 wherein the reentrant region comprises a closed pathway on the fibrillating heart.

25. A method according to claim 24 wherein a wavefront propagates along the closed pathway from a starting point on the closed pathway to an ending point on the closed pathway.

26. A method according to claim 25 wherein the starting point and the ending point are adjacent to one another on the closed pathway.

27. A method for reducing an occurrence of fibrillation of a heart, comprising:
during heart activity characterized by at least one of normal heartbeat activity, premature heartbeat activity, or nonsustained tachycardia activity, applying an electrical stimulus to a region of the heart containing a fastest activating region.

28. A method according to claim 27 wherein the fastest activating region comprises a reentrant region having a refractory period that is less than areas adjacent to the reentrant region.

29. A method according to claim 27 wherein a first wavefront propagates along a closed pathway on the heart, wherein the first wavefront generates at least a second wavefront that propagates on the heart outside the fastest activating region.

30. A method according to claim 27 wherein the reentrant region comprises a closed pathway on the heart.

31. A method according to claim 28 wherein a wavefront propagates along the closed pathway from a starting point on the closed pathway to an ending point on the closed pathway.

32. A method according to claim 29 wherein the starting point and the ending point are adjacent to one another on the closed pathway.

33. A method according to claim 27 wherein a location of the fastest activating region is determined by:
determining a refractory period associated with the heart using premature stimulation.

34. A method according to claim 28 wherein a location of the fastest activating region is determined by:
determining an activation recovery interval measurement associated with the heart.

35. A method according to claim 27 wherein a location of the fastest activating region is determined by:
determining a Monophasic activation potential (MAP) reading of the heart.

36. A method according to claim 27 wherein a location of the fastest activating region is determined by:
inducing fibrillation of the heart; and
determining at least one of a monophasic activation potential (MAP) reading associated with the fibrillating heart, a refractory period associated with the heart using premature stimulation, and a power spectrum analysis to provide a spectrum with a peak power at a highest frequency.

37. A system for defibrillating a heart in fibrillation, comprising:
means for detecting fibrillation of the heart; and
means for applying a defibrillation stimulus to a fastest activating region of the fibrillating heart.

38. A system according to claim 37 wherein the fastest activating region comprises a reentrant region having a refractory period that is less than areas adjacent to the reentrant region.

39. A system according to claim 37 wherein a first wavefront propagates along a closed pathway on the fibrillating heart, wherein the first wavefront generates at least a second wavefront that propagates on the fibrillating heart outside the fastest activating region.

40. A system according to claim 37 wherein the fastest region comprises a closed pathway on the fibrillating heart.

41. A system according to claim 38 wherein a wavefront propagates along the closed pathway from a starting point on the closed pathway to an ending point on the closed pathway.

42. A system according to claim 41 wherein the starting point and the ending point are adjacent to one another on the closed pathway.

43. A system according to claim 37:
wherein the fibrillation comprises atrial fibrillation; and
wherein the fastest activating region comprises at least one of adjacent to pulmonary veins of the fibrillating heart and between the pulmonary veins and a left atrial appendage of the fibrillating heart.

44. A system according to claim 37:
wherein the fibrillation comprises ventricular fibrillation; and
wherein the fastest activating region comprises a base of a left ventricle of the fibrillating heart.

45. A system according to claim 37:
wherein the fibrillation comprises ventricular fibrillation; and
wherein the fastest activating region comprises a septum of the fibrillating heart.

46. A system according to claim 37 wherein the means for applying comprises means for applying a first defibrillation stimulus at least one of before, during, or after a second defibrillation stimulus that is greater than the first defibrillation stimulus.

47. A system according to claim 46 wherein at least one of the first and second defibrillation stimuli is applied using at least a pair of electrodes, wherein the pair of electrodes is located one of:
inside the fastest activating region;
outside the fastest activating region; and
a first electrode of the pair of electrodes is inside the fastest activating region and a second electrode of the pair of electrodes is outside the fastest activating region.

48. A method according to claim 37 wherein the defibrillation stimulus is applied using a pair of electrodes, wherein the pair of electrodes is located one of:
inside the fastest activating region;
outside the fastest activating region; and
a first electrode of the pair of electrodes is inside the fastest activating region and a second electrode of the pair of electrodes is outside the fastest activating region.

49. A system according to claim 37 further comprising:
means for applying at least one pacing stimulus to the fastest activating region simultaneously with the defibrillation stimulus.

50. A system according to claim 37 further comprising:
means for applying at least one pacing stimulus to the fastest activating, region immediately before or after the defibrillation stimulus.

51. A system according to claim 37 further comprising:
means for applying at least one first pacing stimulus at the fastest activating region simultaneously with the defibrillation stimulus; and
means for applying at least one second pacing stimulus to the fibrillating heart at a location spaced-apart from the fastest activating region.

52. A system according to claim 51 wherein the at least one second pacing stimulus is applied using at least one line electrode.

53. A system according to claim 37 wherein the region comprises the septum.

54. A system according to claim 37 wherein a location of the fastest activating region is determined by:
determining a Monophasic activation potential (MAP) reading associated with the fibrillating heart.

55. A system for reducing an occurrence of fibrillation of a heart, comprising:
means for detecting a premature contraction of the heart for a plurality of heart beats characterized by nonsustained tachycardia; and
means for applying an electrical stimulus to a region of the heart not in fibrillation likely to contain a fastest activating region.

56. A system according to claim 55 further comprising:
means for inducing fibrillation of the heart; and
means for determining at least one of a monophasic activation potential (MAP) reading associated with the fibrillating heart, a refractory period associated with the heart using premature stimulation, and a power spectrum analysis to provide a spectrum with a peak power at a highest frequency.

57. A system according to claim 55 wherein the fastest activating region comprises a reentrant region having a refractory period that is less than areas adjacent to the reentrant region.

58. A system according to claim 55 wherein a first wavefront propagates along a closed pathway on the heart, wherein the first wavefront generates at least a second wavefront that propagates on the heart outside the fastest activating region.

59. A system according to claim 55 wherein the reentrant region comprises a closed pathway on the heart.

60. A system according to claim 59 wherein a wavefront propagates along the closed pathway from a starling point on the closed pathway to an ending point on the closed pathway.

61. A system according to claim 60 wherein the starting point and the ending point are adjacent to one another on the closed pathway.

62. A system for reducing an occurrence of fibrillation of a heart, comprising:
means for applying, during fibrillation during heart activity characterized by at least one of normal heartbeat activity, premature heartbeat activity, or nonsustained tachycardia activity, an electrical stimulus to a region of a heart that is likely to contain a fastest activating region of the heart.

63. A system according to claim 62 wherein the fastest activating region comprises a reentrant region having a refractory period that is less than areas adjacent to the reentrant region.

64. A system according to claim 63 wherein a first wavefront propagates along a closed pathway on the fibrillating heart, wherein the first wavefront generates at least a second wavefront that propagates on the heart outside the fastest activating region.

65. A system according to claim 62 wherein the reentrant region comprises a closed pathway on the heart.

66. A system according to claim 65 wherein a wavefront propagates along the closed pathway from a starting point on the closed pathway to an ending point on the closed pathway.

67. A system according to claim 66 wherein the starting point and the ending point are adjacent to one another on the closed pathway.

68. A system according to claim 62 further comprising:
means for determining a refractory period associated with the heart using premature stimulation.

69. A system according to claim 62 further comprising:
means for determining an activation recovery interval measurement associated with the heart.

70. A system according to claim 62 further comprising:
determining a Monophasic activation potential (MAP) reading of the heart.

71. A system according to claim 62 further comprising:
means for inducing fibrillation of the heart; and
means for determining a refractory period associated with the heart using premature stimulation.

72. A system according to claim 62 further comprising:
means for inducing fibrillation of the heart; and
means for determining at least one of a monophasic activation potential (MAP) reading associated with the fibrillating heart, a refractory period associated with the heart using premature stimulation, and a power spectrum analysis to provide a spectrum with a peak power at a highest frequency.

73. A computer program product for defibrillating a heart in fibrillation, comprising:
a computer readable medium having computer readable program code embodied therein, the computer readable program code comprising:
computer readable program code configured to detect fibrillation of the heart; and
computer readable program code configured to apply a defibrillation stimulus to a fastest activating region of the fibrillating heart.

74. A computer program product according to claim 73 wherein the fastest activating region comprises a reentrant region having a refractory period that is less than areas adjacent to the reentrant region.

75. A computer program product according to claim 73 wherein a first wavefront propagates along a closed pathway on the fibrillating heart, wherein the first wavefront generates at least a second wavefront that propagates on the fibrillating heart outside the fastest activating region.

76. A computer program product according to claim 73 wherein the fastest region comprises a closed pathway on the fibrillating heart.

77. A computer program product according to claim 76 wherein a wavefront propagates along the closed pathway from a starting point on the closed pathway to an ending point on the closed pathway.

78. A computer program product according to claim 77 wherein the starting point and the ending point are adjacent to one another on the closed pathway.

79. A computer program product according to claim 73:
wherein the fibrillation comprises atrial fibrillation; and
wherein the fastest activating region comprises at least one of adjacent to pulmonary veins of the fibrillating heart and between the pulmonary veins and a left atrial appendage of the fibrillating heart.

80. A computer program product according to claim 73:
wherein the fibrillation comprises ventricular fibrillation; and
wherein the fastest activating region comprises a base of a left ventricle of the fibrillating heart.

81. A computer program product according to claim 73:
wherein the fibrillation comprises ventricular fibrillation; and
wherein the fastest activating region comprises a septum of the fibrillating heart.

82. A computer program product according to claim 73 wherein the computer readable program code configured to apply comprises computer readable program code configured to apply a first defibrillation stimulus at least one of before, during, or after a second defibrillation stimulus that is greater than the first defibrillation stimulus.

83. A computer program product according to claim 82 wherein at least one of the first and second defibrillation stimuli is applied using at least a pair of electrodes, wherein the pair of electrodes Is located one of:
inside the fastest activating region;
outside the fastest activating region; and
a first electrode of the pair of electrodes is inside the fastest activating region and a second electrode of the pair of electrodes is outside the fastest activating region.

84. A method according to claim 73 wherein the defibrillation stimulus is applied using a pair of electrodes, wherein the pair of electrodes is located one of:
inside the fastest activating region;
outside the fastest activating region; and
a first electrode of the pair of electrodes is inside the fastest activating region and a second electrode of the pair of electrodes is outside the fastest activating region.

85. A computer program product according to claim 73 further comprising:
computer readable program code configured to apply at least one pacing stimulus to the fastest activating region simultaneously with the defibrillation stimulus.

86. A computer program product according to claim 73 further comprising:
computer readable program code configured to apply at least one pacing stimulus to the fastest activating region immediately before or after the defibrillation stimulus.

87. A computer program product according to claim 73 further comprising:
computer readable program code configured to apply at least one first pacing stimulus at the fastest activating region simultaneously with the defibrillation stimulus; and
computer readable program code configured to apply at least one second pacing stimulus to the fibrillating heart at a location spaced-apart from the fastest activating region.

88. A computer program product according to claim 87 wherein the at least one second pacing stimulus is applied using at least one line electrode.

89. A computer program product according to claim 73 wherein the region comprises the septum.

90. A computer program product according to claim 73 wherein the computer readable program code configured determine the location of the fastest activating region comprises:
computer readable program code configured to determine a Monophasic activation potential (MAP) reading associated with the fibrillating heart.

91. A computer program product for reducing an occurrence of fibrillation of a hearts, comprising:
a computer readable medium having computer readable program code embodied therein, the computer readable program code comprising:
computer readable program code configured to detect a premature contraction of the heart for a plurality of heart beats characterized by nonsustained tachycardia; and
computer readable program code configured to apply a defibrillation stimulus to a region of the heart not in fibrillation that is likely to contain a fastest activating region.

92. A computer program product according to claim 91 further comprising computer readable program code configured to, determine the region of the heart likely to contain the fastest-activating region including:
computer readable program code configured to induce fibrillation of the heart; and
computer readable program code configured to determine at least one of a monophasic activation potential (MAP) reading associated with the fibrillating heart, a refractory period associated with the heart using premature stimulation, and a power spectrum analysis to provide a spectrum with a peak power at a highest frequency.

93. A computer program product according to claim 91 wherein the fastest activating region comprises a reentrant region having a refractory period that is less than areas adjacent to the reentrant region.

94. A computer program product according to claim 91 wherein a first wavefront propagates along a closed pathway on the fibrillating heart, wherein the first wavefront generates at least a second wavefront that propagates on the fibrillating heart outside the fastest activating region.

95. A computer program product according to claim 91 wherein the reentrant region comprises a closed pathway on the fibrillating heart.

96. A computer program product according to claim 95 wherein a wavefront propagates along the closed pathway from a starting point on the closed pathway to an ending point on the closed pathway.

97. A computer program product according to claim 96 wherein the starting point and the ending point are adjacent to one another on the closed pathway.

98. A computer program product for reducing an occurrence of fibrillation of a heart, comprising:
a computer readable medium having computer readable program code embodied therein, the computer readable program code comprising:
computer readable program code configured to apply, during heart activity characterized by at least one of normal heartbeat activity, premature heartbeat activity, or nonsustained tachycardia activity, an electrical stimulus to a region of the heart not in fibrillation that is likely to contain a fastest activating region.

99. A computer program product according to claim 98 wherein the fastest activating region comprises a reentrant region having a refractory period that is less than areas adjacent to the reentrant region.

100. A computer program product according to claim 98 wherein a first wavefront propagates along a closed pathway on the fibrillating heart, wherein the first wavefront generates at least a second wavefront that propagates on the fibrillating heart outside the fastest activating region.

101. A computer program product according to claim 98 wherein the reentrant region comprises a closed pathway on the fibrillating heart.

102. A computer program product according to claim 101 wherein a wavefront propagates along the closed pathway from a starting point on the closed pathway to an ending point on the closed pathway.

103. A computer program product according to claim 102 wherein the starting point and the ending point are adjacent to one another on the closed pathway.

104. A computer program product according to claim 98 further comprising computer readable program code configured to determine the region of the heart likely to contain the fastest activating region including:
computer readable program code configured to determine a refractory period associated with the fibrillating heart using premature stimulation.

105. A computer program product according to claim 98 further comprising computer readable program code configured to determine the region of the heart likely to contain the fastest activating region including:
computer readable program code configured to determine an activation recovery interval measurement associated with the fibrillating heart.

106. A computer program product according to claim 98 further comprising computer readable program code configured to determine the region of the heart likely to contain the fastest activating region including:
computer readable program code configured to determining a Monophasic activation potential (MAP) reading associated with the fibrillating heart.

107. A computer program product according to claim 98 further comprising computer readable program code configured to determine the region of the heart likely to contain the fastest activating region including:
computer readable program code configured to induce fibrillation of the heart; and
computer readable program code configured to determine at least one of a monophasic activation potential (MAP) reading associated with the fibrillating heart, a refractory period associated with the heart using premature stimulation, and a power spectrum analysis to provide a spectrum with a peak power at a highest frequency.
